(19) United States
(12) Patent Application Publication
(10) Pub. No.: US 2013/0178603 A1
(43) Pub. Date: Jul. 11, 2013

(54) MULTI-MODE CANCER TARGETED NANOPARTICULATE SYSTEM AND A METHOD OF SYNTHESIZING THE SAME

(71) Applicants: Rassoul Dinarvand, Tehran (IR); Mohammad Ali Derakhshan, Tehran (IR); Fatemeh Rahbarizadeh, Tehran (IR); Reza Faridi Majidi, Tehran (IR); Azade Taheri Borujeni, Tehran (IR); Seyed Mahdi Rezayat, Tehran (IR)

(72) Inventors: Rassoul Dinarvand, Tehran (IR); Mohammad Ali Derakhshan, Tehran (IR); Fatemeh Rahbarizadeh, Tehran (IR); Reza Faridi Majidi, Tehran (IR); Azade Taheri Borujeni, Tehran (IR); Seyed Mahdi Rezayat, Tehran (IR)

(21) Appl. No.: 13/347,813

(22) Filed: Jan. 11, 2012

(51) Int. Cl. C07K 17/02; B82Y 5/00

(52) U.S. Cl. 530/364; 530/363; 977/773; 977/906

(57) ABSTRACT

The various embodiments herein provide a method of synthesizing a multi-mode cancer targeted nanoparticles. The method comprises the steps of preparing a plurality of nanoparticles and covalently conjugating monoclonal antibodies on surface of the prepared plurality of nanoparticles. The plurality of nanoparticles consists of a protein and a drug. The protein is Human Serum Albumin protein (HSA) and the drug is methotrexate. The monoclonal antibodies are anti-MUC1 nanobodies. According to an embodiment herein, a multi-mode cancer targeted nanoparticles comprising a plurality of cross linked nanoparticles of protein and drug molecules and covalently linked molecules of monoclonal antibodies. The molecules of monoclonal antibodies are linked on a surface of the plurality of cross linked nanoparticles.

### BACKGROUND

1. Technical Field

The embodiments herein generally relate to the drug delivery systems and more particularly to a targeted drug delivery system for a treatment of cancer. The embodiments herein also relate to a method of making the targeted drug delivery system for the cancer treatment.

2. Description of the Related Art

The recent striking progress in nano-oncology holds a great promise to diagnose the cancerous cells early and treat them efficiently without any notable destructive effects on the other healthy cells. Based on nano-technological procedures, different delivery systems have been introduced. A vast variety of synthetic and natural polymers can be used for a nanoparticle preparation. Among these, Human Serum Albumin (HSA), as a natural polymer, has extensively been studied for the preparation of micro and nano particles.

Versatility of albumin as a protein carrier for drug targeting and improving the pharmacokinetic profile of peptide or protein-based drugs is well understood. Albumin is synthesized in the liver and with the concentration of 35-50 g/L in human serum, is the most abundant plasma protein. This protein has a molecular weight of 66.5 kDa. The Human Serum Albumin (HSA) shows an average half-life of 19 days. The most important characteristic of the albumin is its ability to bind a great number of therapeutic drugs and molecules in the blood. It is noteworthy to mention that albumin is considered as a very soluble protein with appreciable stability in harsh conditions such as in the pH range of 4-9 and at a heating process at 60° C. for up to 10 h without any destructive effects. Beside these properties, a specific accumulation in tumor and inflamed tissues, the biodegradability and a lack of toxicity, make albumin an appropriate natural polymer for drug delivery applications.

It is revealed that the albumin accumulates in a malignant tissue due to the presence of leaky capillaries associated with an impaired lymphatic drainage system. This phenomenon is known as an enhanced permeability and retention (EPR) effect.

The first experience of drug conjugation to HSA with phases I/II clinical studies was on Methotrexate-Albumin conjugate (MTX-HSA). In this strategy a drug was attached to lysine residues of HSA. The MTX as an anti-cancer drug is a folate antagonist that is effective on choriocarcinoma. The mean distribution half-life of MTX is short (1.5-3.5 hrs) so it is rapidly cleared from circulation through the kidneys after I.V administration. Consequently, the success of a drug delivery to the tumor decreases. In a study, a formulation of the MTX in bovine serum albumin nano particles has been reported. The absence of a targeting moiety in this system may induce side effects. Recently, Taheri et al. prepared a HSA-based nanoparticle system containing conjugated MTX. In this strategy, they used N-ethyl-N-(3-dimethylaminopropyl) carbodiimide HCl (EDC) as the cross-linking agent in 2 stages. As reported in studies, the reactive intermediate of EDC is water soluble and could easily been hydrolyzed.

The active targeting strategies involve conjugation of targeting molecules to the surface of nano particles to increase an internalization of nanoparticle systems into the desired cells. During a long circulation of the blood, nano-particles find their ways to the tumor site through the EPR effect, and the targeting molecule can enhance endocytosis of the nano-particles. Several attempts have been made to use antibodies and small molecules as targeting moieties. However, immunogenicity is a limiting factor in the use of the antibodies for therapeutic purposes.

Hamers-Casterman et al. in 1993 reported the discovery of new kind of antibodies in camelids that lack light chains and their single N-terminal domain (VHH or NANOBODY®) binds antigen effectively. Compared to a conventional antibody (150 kDa), such as an antigen-binding fragment or Fab (55 kDa) and a single chain variable fragment or scFv (30 kDa), the nano-bodies are the smallest functional antibody fragments (15 kDa). Also, the nano-bodies show a high stability against a heat-denaturation and present a better hydrophilicity than Fabs and scFvs. Moreover, the capability to be easily engineered and produced in bacteria or yeast, make the nano-bodies as promising moieties for the biomedical applications. But, none of the prior arts uses an anti-MUC1 nanobody as a targeting molecule against the MUC1 glycoprotein on the cancer cell surfaces.

Hence, there is a need to provide a successful cancer targeted conjugated moieties that are very effective and have no side effects.

The above-mentioned shortcomings, disadvantages and problems are addressed herein and which will be understood by reading and studying the following specification.

### OBJECTIVES OF THE EMBODIMENTS

The primary object of the embodiments herein is to provide a multi-mode cancer targeted nano-particles conjugated with anti-MUC1 nano-body as a targeting molecule against MUC1 glycoprotein on cancer cell surfaces.

Another object of the embodiments herein is to provide a biodegradable multi-mode cancer targeted nanoparticles conjugated with anti-MUC1 nanobody.

Yet another object of the embodiments herein is to provide a multi-mode cancer targeted nanoparticles conjugated with anti-MUC1 nanobody that reaches and internalizes to the tumor cells through the passive and active strategies.

Yet another object of the embodiments herein is to provide a multi-mode cancer targeted nanoparticles conjugated with anti-MUC1 nanobody that has lower cytotoxicity to the non-cancerous cells.

These and other objects and advantages of the embodiments herein will become readily apparent from the following detailed description taken in conjunction with the accompanying drawings.

### SUMMARY

The various embodiments herein provide a method of synthesizing a multi-mode cancer targeted nanoparticles. According to an embodiment herein, a method of synthesizing a multi-mode cancer targeted nanoparticles comprising the steps of preparing a plurality of nanoparticles and covalently conjugating monoclonal antibodies on surface of the prepared plurality of nanoparticles. The plurality of nanoparticles consists of a protein and a drug. The protein and the drug are cross-linked to each other. The protein is a Human Serum Albumin protein (HSA). The drug is selected from a group consisting of melphalan, doxorubicin, daunorubicin, gemcitabine and methotrexate, and wherein the drug is methotrexate (MTX). Each of the plurality of nanoparticles including Human Serum Albumin protein and methotrexate is MTX-HSA. The monoclonal antibodies are anti-MUC1 nanobodies (VHH). The multi-modal cancer targeted nanoparticles include the Human Serum Albumin protein, the methotrexate and the monoclonal antibody. The multi-modal cancer targeted nanoparticles comprising the Human Serum Albumin protein, the methotrexate and the monoclonal antibody is MTX-HSA-VHH.

The step of preparing the plurality of nanoparticles further comprises solubilizing a drug in a solvent with N-ethyl-N-(3-dimethylaminopropyl) carbodiimide HCl (EDC) and N-hydroxy succinimide (NHS) to form a first solution. The solvent is Dimethyl sulfoxide (DMSO). The first formed solution is incubated in a water bath at 60° C. for 10 min. The incubated solution is cooled to a room temperature. The cooled solution is added drop wise to a solution of protein while stirring at 400 rpm for 10 min to obtain a second solution. The solution of protein is prepared by dissolving the Human Serum Albumin (HSA) in a phosphate buffer saline. The obtained second solution is dialyzed against the phosphate buffer saline (PBS) by using a dialysis tube for 24 h. The dialyzed solution is lyophilized at −40° C. for 24 h and a third solution containing the plurality of nanoparticles is obtained.

The step of covalently conjugating the monoclonal antibodies on a surface of the prepared plurality of nanoparticles further comprises solubilizing the obtained nanoparticles, N-ethyl-N-(3-dimethylaminopropyl) carbodiimide HCl (EDC) and N-hydroxy succinimide (NHS) in a phosphate buffer saline to form a reaction mixture. The reaction mixture is stirred at 200 rpm for 15 min at a room temperature. The stirred reaction mixture is ultra filter centrifuged by a 30 kDa ultra filter device and centrifuged at 5000 g for 10 min. Then the solubilized nanoparticles are obtained. The obtained nanoparticles are resuspended in 1 ml of the phosphate buffer saline (PBS) to obtain a solution. The pH of the PBS is kept at 7.4. The monoclonal antibodies are introduced into the obtained solution to form a mixture. The monoclonal antibodies are anti-MUC1 nanobodies. The formed mixture is stirred at 200 rpm for 2 hrs at a room temperature. The stirred solution is ultrafilter centrifuged using a 30 kDa ultrafilter device and centrifuged at 5000 g for 10 min. Finally, the targeted protein based conjugated nanoparticles are obtained.

According to one embodiment herein, the protein is a denatured protein. The denatured protein is obtained using a denaturant. The denaturant is Dimethyl sulfoxide (DMSO).

The MTX-HSA nanoparticles have a particle size of about 90-150 nm. The VHH-MTX-HSA nanoparticles are spherical in shape with a hydrodynamic size in the range of 100-200 nm in diameter. The VHH-MTX-HSA nanoparticles have a hydrodynamic size of 40 nm in diameter. The MTX-HSA nanoparticles have a zeta-potential of about −20 mv. The VHH-MTX-HSA nanoparticles have a zeta-potential in a range of −10 mv to −20 mv. The VHH-MTX-HSA nanoparticles have a zeta-potential of −10 mv.

The plurality of nanoparticles has a particle size of about 90-150 nm and the covalently conjugated monoclonal antibodies on the surface of nanoparticles has a particle size of 100-200 nm.

According to an embodiment herein, a multi-mode cancer targeted nanoparticles comprising a plurality of cross-linked nanoparticles of protein and drug molecules and covalently linked molecules of monoclonal antibodies. The molecules of monoclonal antibodies are linked on a surface of the plurality of cross-linked nanoparticles. The protein is a Human Serum Albumin (HSA). The drug includes an anticancer drug containing carboxyl or primary amino functional groups and the drug is methotrexate. The drug is selected from a group consisting of melphalan, doxorubicin, daunorubicin, gemcitabine and methotrexate. The drug is methotrexate (MTX). The monoclonal antibody is a single N-terminal domain, and the monoclonal antibody is anti MUC1 (mucin glycoprotein) i.e. VHH. The system has an active and passive tumor targeting capability. The cancer treated by the nanoparticles according to the embodiments herein is a kind of cancer that over-expresses mucin glycoproteins (MUC1) on cell surfaces. The cancer is carcinoma. The MTX-HSA nanoparticles have a particle size of about 90-150 nm. The VHH-MTX-HSA nanoparticles are spherical in shape with a hydrodynamic size in the range of 100-200 nm in diameter. The VHH-MTX-HSA nanoparticles have a hydrodynamic size of 40 nm in diameter. The MTX-HSA nanoparticles have a zeta-potential of about −20 mv. The VHH-MTX-HSA nanoparticles have a zeta-potential in a range of −10 mv to −20 mv. The VHH-MTX-HSA nanoparticles have a zeta-potential of −10 mv.

The plurality of nanoparticles has a particle size of about 90-150 nm and the covalently linked monoclonal antibodies on the surface of the nanoparticles has a particle size of 100-200 nm. The plurality of nanoparticles has a zeta-potential of about −20 mv and the covalently linked monoclonal antibodies on the surface of nanoparticles have a zeta-potential of −10 mv.

The drug has an efficiency of 48% and wherein the efficiency of monoclonal antibody conjugation is 80%. The nanoparticles are spherical and about 40 nm in diameter and their hydrodynamic size is in the range of 100-200 nm in diameter.

According to an embodiment, a method of producing bioconjugated methotrexate-Human serum albumin (MTX-HSA) nanoparticles comprises preparing nanobody-methotrexate-human serum albumin conjugate (VHH-MTX-HAS) nanoparticles via covalent attachment, wherein the HSA molecules are denatured and covalently linked to each other.

According to one embodiment herein, a method of producing targeted albumin-based nanoparticles for anti-cancer drug delivery is provided. The method comprises the steps of forming a plurality of nanoparticles of albumin and drug. The plurality of nanoparticles of albumin and drug is formed using a desolvation process. The formed plurality of nanoparticles of albumin and drug are simultaneously cross linked to the monoclonal antibodies by adding a cross linker for a covalent attachment of the monoclonal antibodies to the surface of nanoparticles. The albumin is a Human Serum Albumin (HSA). The anti-cancer drug is methotrexate (MTX) or an anti-cancer drugs containing carboxyl or primary amino functional groups. The plurality of nanoparticles of albumin and drug is referred herein as MTX-HSA. The type of cancer treated is the cancer in which MUC1 glycoprotein is over-expressed on the cell surfaces. The cancer is a carcinoma. The MTX-HSA nanoparticles formed via using Dimethyl sulfoxide (DMSO) and N-ethyl-Ń-(3-dimethylaminopropyl) carbodiimide HCl (EDC) and N-hydroxy succinimide (NHS) chemistry. The DMSO is the solvent for the MTX and the protein (albumin) denaturant. The HSA molecules are denatured and covalently linked to each other. The MTX molecules are encapsulated and covalently linked to the HSA molecules. The monoclonal antibody is a single N-terminal domain (VHH or NANOBODY®). The nanobody is an anti-MUC1 VHH. The nanobody molecules are covalently attached to the MTX-HSA nanoparticle via EDC/NHS chemistry and form a VHH-MTX-HSA nanoparticle. The nanoparticles are spherical and about 40 nm in diameter and hydrodynamic size is in the range of 90-150 nm. The zeta-potential of said nanoparticles is about −20 mv. The hydrodynamic size of the nanoparticles is about 100-200 nm in diameter. The zeta-potential of the nanoparticles is about −10 mv. The drug loading efficiency is about 48%. The nanobody conjugation efficiency is more than 80%.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 shows a schematic representation of the formation of Methotrexate-Human Serum Albumin (MTX-HSA) nanoparticles, according to an embodiment herein.

FIG. 2 shows a schematic representation of the covalent attachment of a nanobody to the MTX-HSA nanoparticles, according to an embodiment herein.

FIG. 3 shows a Transmission Electron Microscopic (TEM) image of the Methotrexate-Human Serum Albumin (MTX-HSA) nanoparticles, according to an embodiment herein.

FIG. 4A shows an electropherogram of the Human Serum Albumin protein, according to an embodiment herein.

FIG. 4B shows an electropherogram of the Methotrexate drug, according to an embodiment herein.

FIG. 4C shows an electropherogram of the Methotrexate-Human Serum Albumin (MTX-HSA) nanoparticles, according to an embodiment herein.

FIG. 5 shows a calibration curve of absorbance of different concentrations of the MTX at 372 nm, according to the embodiments herein.

FIG. 6 shows a standard curve of absorbance of the concentrations of the BSA ranging from 10 to 100 μg/ml at 595 nm, according to the embodiments herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments herein are described in sufficient detail to enable those skilled in the art to practice the embodiments herein and it is to be understood that the logical, mechanical and other changes may be made without departing from the scope of the embodiments herein. The following detailed description is therefore not to be taken in a limiting sense.

The various embodiments herein provide a smart cancer targeting nanoparticle drug delivery system and its preparation. The smart cancer targeting nanoparticle drug delivery system is an albumin based system. The drug delivery system is made up of drug and protein nanoparticles conjugated with nanobodies on their surfaces. The drug includes any anticancer drug containing carboxyl or primary amino functional groups. The nanobodies are covalently attached to the surface of the nanoparticles to achieve an active targeting nano-particulate drug delivery system.

According to a preferred embodiment herein, the nanoparticles comprise methotrexate as an anti cancer drug and a Human Serum Albumin (HSA) as a protein, conjugated with the anti MUC1 nanobodies. The cancer targeted nanoparticles disclosed in the embodiments herein, is capable of passive and active tumor targeting. According to an embodiment herein, an anti-MUC1 nanobody is an anti-MUC1 VHH.

According to an embodiment herein, the drug includes the anti-cancer drugs such as Melphalan, Doxorubicin, Daunorubicin, Gemcitabine and methotrexate. Preferably the drug is methotrexate. In the case of the primary amine containing drugs, the carboxyl groups of the HSAs is activated by EDC/NHS chemistry and then the amide bond is formed between these activated carboxyl groups and primary amines of the drugs.

The MUC1 gene encodes a mucin glycoprotein. MUC1 is, normally, expressed on the apical surface of mammary epithelial cells. However, in a breast adenocarcinoma and a number of epithelial tumors, it is over-expressed on the entire cell surface. This property makes the MUC1 protein as a valuable biomarker in a breast cancer treatment. Also, the studies show that MUC1 is expressed in the hematopoietic cells, the T- and B-lymphocytes, the hepatocytes, the myocytes and the nerve cells.

A passive targeting scheme is one of the common strategies that are followed by nanotechnologists to design a cancer drug delivery system. It is noteworthy to mention that the tumor tissue has an impaired structure (fenestrated endothelium of vasculatures and non-effective lymphatic drainage) that helps the tumor to get more nutrients for a growth and makes an efficient route to deliver the drugs into the tumor site. Basically, the blood macromolecules and the drug loaded nano-particulate systems can find their ways through the big pores in the cancer vasculature and be sequestered and accumulated at the tumor tissue. As this process is caused by the blood circulation, this is called passive targeting scheme.

Although the passive targeting strategy can help the nanoparticle systems to accumulate at the tumor site but an effective internalization of the nanoparticles into the cancer cells requires the special moieties that can recognize and attach the specific receptors on the cell surfaces. The researchers have utilized different targeting molecules (antibodies, antibody fragments, small molecules) to decorate the nanoparticle surfaces. These targeting molecules can find their receptors on the cancer cell surface and trigger a subsequent internalization of the nanoparticle into the cell. This kind of targeting is called an active targeting scheme.

The Enhanced Permeation and Retention effect (EPR) is caused by the impaired structure of the tumor tissue (unusual fenestrated vasculatures and the weak lymphatic drainage). This phenomenon leads to an enhanced permeation of the blood macromolecules and the nanoparticles into the tumor tissue through the porous vasculatures. Also, the weak lymphatic drainage originates an additional retention of the molecules and the particles at the tumor site.

The multi-mode refers to the different capabilities of the nanoparticulate system to target the tumor tissue in different modes i.e. methods of action. In fact, the multi-mode system provides the associated passive and active targeting mechanisms to efficiently attack the cancer tissue and cells. Also, the active targeting ability, in turn, includes the advantages of two different targeting moieties. In this respect, an anti-MUC1 VHH, the special decoration on the multi-mode nanoparticle system, can target MUC1 receptors on the cancer cell surfaces. The MTX drug which is somewhat available at the nanoparticle surface can be an additional targeting molecule. The MTX can recognize and attach to the folate receptors on the tumor cell surfaces and give rise to the cellular internalization of the nanoparticles.

There are three mechanisms of action for the multi-mode nanoparticles. Basically, a passive accumulation and sequestration of the drug loaded nanoparticles at the tumor site occurs through the EPR effect after an intravenous administration. However, an active targeting and internalization of the particles are led by two different moieties (anti-MUC1 VHH and MTX) available on the nanoparticle surface. The binding of the anti-MUC1 VHH to its receptor triggers internalization of the nanoparticles into the cells. These mechanisms support the site-specific delivery of the cancer drug and therefore, decrease the side effects of the MTX on the healthy cells. Also a presence of the MTX molecules on the surface of the nanoparticles provides for an additional active targeting opportunity through an attachment of the MTX molecules to the folate receptors. Both the active targeting molecules promote the receptor-dependant endocytosis of the nanoparticles. Consequently, the nanoparticle structure is destroyed in the lysosome and the drug payload is released into the cytosol to take action. At this stage, the MTX arrests the cellular metabolism and causes cancer cell death.

According to an embodiment herein, a method of preparing a multi-mode cancer targeted nanoparticles comprises the steps of preparing a plurality of nanoparticles and covalently conjugating nanobodies on the surface of the prepared plurality of nanoparticles. The plurality of nanoparticles consists of a protein and a drug. The protein is a Human Serum Albumin protein (HSA) and the drug is a methotrexate. The protein and the drug are first cross linked to each other to form a conjugate referred as MTX-HSA and then covalently attached to the nanobodies.

The step of preparation of MTX-HSA nanoparticles further comprises the steps of first solubilizing 20 mg of methotrexate drug, 25 mg N-ethyl-N-(3-dimethylaminopropyl) carbodiimide HCl (EDC) and 60 mg N-hydroxy succinimide (NHS) in 1 ml of Dimethyl sulfoxide (DMSO) solvent. The solution is incubated in a water bath at 60° C. for 10 min. The incubated solution is cooled to a room temperature and further 5 ml of phosphate buffer saline (PBS) solution is added dropwise to a solution of 250 mg of HSA while stirring the solution at 400 rpm for 10 min. The PBS is used with a concentration of 0.02 M and at a pH of 7.4. The obtained solution dialyzed against the PBS by using a dialysis tube (cellulose membrane at cut-off 12000 kDa from Merck, Germany) for 24 h. Further, a lyophilizing of the solution containing MTX-HSA nanoparticles is done at −40° C. for 24 h.

The step of covalently conjugating nanobodies on surface of the prepared plurality of nanoparticles further comprises solubilizing 5 mg of MTX-HSA nanoparticles, 1 mg of EDC and 2.5 mg of NHS in 1 ml PBS. The pH of PBS is kept at 7.4. After stirring the reaction mixture at 200 rpm for 15 min at room temperature, an ultrafilter centrifuging of the solution is performed by a 30 kDa ultrafilter device (AMICON Ultra-30kDa) and the centrifuging of the solution is carried out at 5000 g for 10 min. The obtained nanoparticles are resuspended in 1 ml of PBS having pH 7.4 and 125 μg of anti-MUC1 VHH is introduced into the solution. Further, the solution is stirred at 200 rpm for 2 hrs at room temperature and ultrafilter centrifuging of the solution is performed using a 30 kDa ultrafilter device and a centrifugation is carried out at 5000 g for 10 min.

In the embodiments herein, the cross-linker agent is N-ethyl-Ń-(3-dimethylaminopropyl) carbodiimide HCl (EDC). N-hydroxy succinimide (NHS) is added to the reaction solution in order to increase the chance of amide bond formation and stabilize the reactive intermediates. The EDC and NHS added herein are utilized in one stage.

According to one embodiment, in the method of preparing the multi-mode cancer targeted nanoparticles, an activation of the MTX carboxyl groups occurs at 60° C. for 10 min. Then, the subsequent nanoparticle preparation is performed at a room temperature. The pH value of the buffer required for the reaction is 7.4. The conjugation is achieved at a room temperature and the pH value of the buffer is 7.4.

The factors that affect the formation and the size distribution of the resultant nanoparticles include an amount of the cross-linker (EDC associated with NHS), a pH of the phosphate buffer used and a duration of the carboxyl group activation. The preferred conditions for MTX-HSA NPs preparation is 5-30 mg EDC and 45-70 mg NHS, pH 7-7.4 and a reaction duration of 10-15 min. The preferred conditions for VHH conjugation to the

MTX-HSA nanoparticles is 1-5 mg EDC, 2.5-12.5 mg NHS, pH 7-7.4 and the activation duration of 15-20 min.

According to the most preferred embodiments herein, the most preferred conditions for MTX-HSA NPs preparation is 25 mg EDC, 60 mg NHS, pH 7.4 and the reaction duration of 10 min. The most preferred conditions for VHH-MTX-HSA NPs preparation is 1 mg EDC, 2.5 mg NHS, pH 7.4 and 15 min of activation reaction.

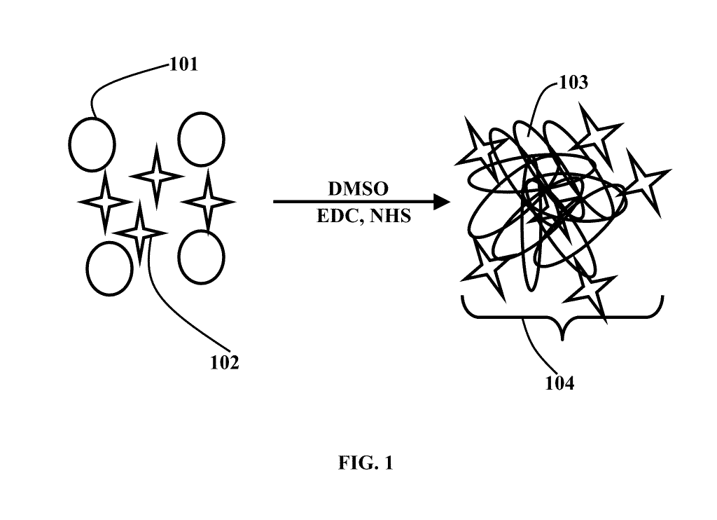

FIG. 1 shows a schematic representation of the formation of Methotrexate-Human Serum Albumin (MTX-HSA) nanoparticles, according to an embodiment herein. As shown in FIG. 1, individual Human Serum Albumin (HSA) proteins molecules 101 participate in the formation of nanoparticles while methotrexate (MTX) molecules 102 conjugate to the HSA surface. The HSA proteins 101 gets denatured in the presence of reagents NHS, EDC and DMSO and form the denatured HSA proteins 103. The conjugate is referred to as Methotrexate-Human Serum Albumin (MTX-HSA) 104. The addition of NHS catalysed by EDC causes the activation of carboxyl groups of MTX molecules 102 by esterification. The dropwise addition of this solution containing DMSO (MTX in DMSO) to the aqueous solution of HSA proteins causes a denaturation of the HSA proteins 101 and their hydrophobic aggregation. Therefore, DMSO acts as a denaturant herein. At the same time, the activated carboxyl groups of the MTX molecules 102 attack the primary amino groups on the HSA molecules 101 and form an amide bond. Also some amount of EDC and NHS reagents activate the carboxyl groups on the protein surface. These activated carboxyl groups, in turn, attacks the amino groups on the other HAS protein molecules and cross-link them to each other. Therefore, simultaneous to the covalent attachment of the MTX 102 to the HSA 101, amide bonds form between carboxyl and amide groups on different HSAs and result in the formation of the MTX-HSA nanoparticles.

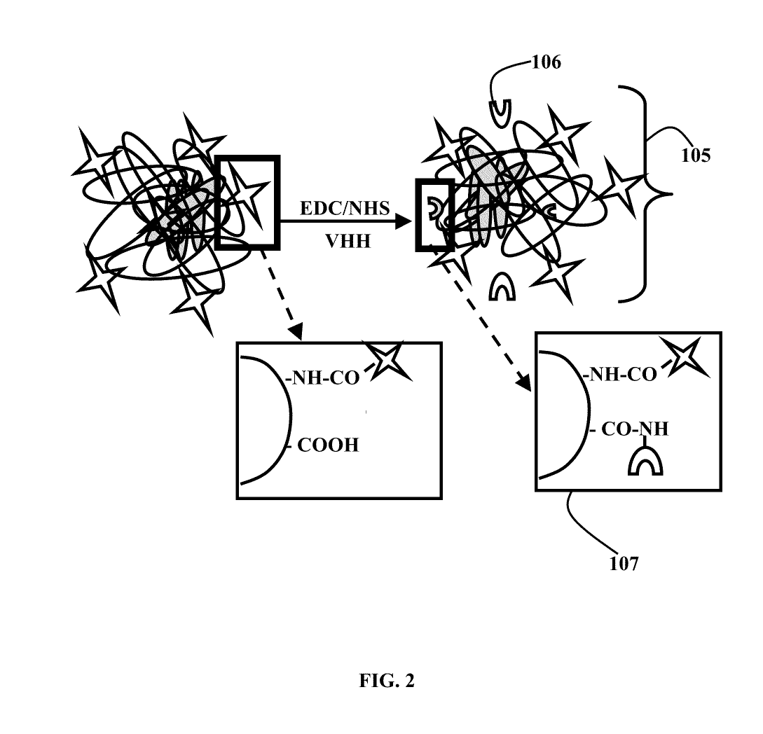

FIG. 2 shows a schematic representation of the covalent attachment of a nanobody to the MTX-HSA nanoparticles, according to an embodiment herein. With respect to FIG. 2, the shaping of the nanobody-MTX-HSA nanoparticles 105 can be seen. In the process of conjugation, the carboxyl groups on the resultant MTX-HSA nanoparticles 104 are activated using EDC and NHS chemistry. After incubation the non-reacted reagents are removed to prevent the increase in the particle size. An ultrafilter device is applied to separate the activated nanoparticles. After a resuspension of the activated nanoparticles solution, the nanobodies (VHH) 106 were added. The nanobody 106 is anti-MUC1 nanobody. The activated carboxyl groups of the nanoparticles attacked the primary amino groups of the nanobodies that led to a covalent attachment of VHHs to the activated carboxyl groups on the nanoparticles 107.

The hydrodynamic size of the MTX-HSA NPs and VHH-MTX-HSA NPs are in the range of 90-150 nm and 100-200 nm, respectively. The zeta-potential of the MTX-HSA NPs and VHH-MTX-HSA NPs are about −20 and −10 mV, respectively. The reduction of the zeta potential in the VHH-MTX-HSA NPs proves the conjugation of the nanobodies to the MTX-HSA NPs.

### EXPERIMENTAL DATA

### Example 1

Materials: Methotrexate USP was kindly donated by Cipla Pharmaceutical Co., India. 1-ethyl-3-(3-dimethylaminopropyl) carbodiimide HCl and human serum albumin (HSA) were purchased from Sigma (Steinheim, Germany). NHS (N-hydroxy succinimide) was purchased from Merck (Darmstadt, Germany). Anti-MUC1 nanobody was prepared at Dr Rabraizadeh's lab (Tarbiat Modares University, Iran). DMSO was purchased from Merck (Darmstadt, Germany). Coomassie blue G250 was purchased from Sigma (Steinheim, Germany). Deionized water was used throughout the experiment. All other chemicals used were of reagent grade.

### Example 2

Preparation of MTX-HSA nanoparticles: A two-step procedure was utilized to simultaneously conjugate MTX to HSA protein and form MTX-HSA NPs. First 20 mg of MTX, 25 mg EDC and 60 mg NHS were solubilised in 1 ml of DMSO. The solution was incubated in a water bath at 60° C. for 10 min. Then the solution was cooled to room temperature and added dropwise to a solution of 250 mg HSA in 5 ml phosphate buffer saline (0.02M, pH 7.4) while stirring at 400 rpm. After 10 min, the obtained solution was dialyzed against PBS by using a dialysis tube (cellulose membrane, cut-off 12000 kDa from Merck, Germany) for 24 h. The resultant nanoparticle solution was lyophilized (at −40° C., 24 h).

### Example 3

Morphology, particle size and zeta potential of the MTX-HSA NPs: 3 mg of the freeze-dried NPs were dispersed in PBS (pH 7.4) then hydrodynamic diameter and zeta potential of the particles were determined by a Zetasizer Nano ZS (Malvern Instruments, Worcestershire, UK). Also the surface morphology of the MTX-HSA NPs was visualized by transmission electron microscopy. A TEM instrument (Ziess EM900, Germany) with an acceleration voltage of 80 KV was utilized.

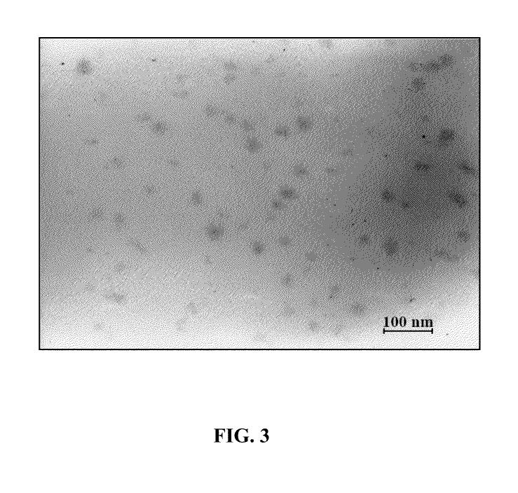

FIG. 3 shows a Transmission Electron Microscopic (TEM) image of the Methotrexate-Human Serum Albumin (MTX-HSA) nanoparticles, according to an embodiment herein. With respect to FIG. 3, the size of Methotrexate-Human Serum Albumin (MTX-HSA) nanoparticles is about 40 nm in diameter.

The electropherograms of the Methotrexate (MTX), the Human Serum Albumin (HSA) protein and the Methotrexate-Human Serum Albumin (MTX-HSA) nanoparticles were calculated. Based on the experience, the best pH value for sample injection into the capillary electrophoresis column was about 2. At this pH, a migration time of the analytes was reduced and their UV absorbance (at 300 nm) was the best. It arises from the fact that this pH is less than isoelectric pH of both HSA (pI 5.3) and MTX (pI 4.7) therefore, they are positively charged and after application of a high voltage of 20 KV, leads to an increased migration rate. Also the fused silica of the column is protonated and, consequently, has no interaction with the analytes. An absorbance measurement was performed at 300 nm as it was found to be the λₘₐₓ of the MTX.

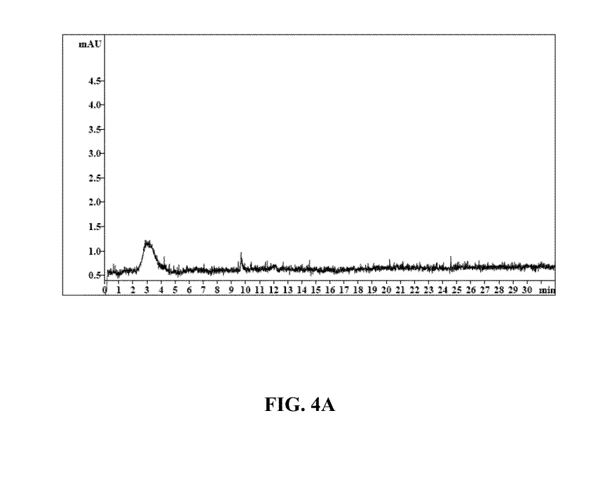

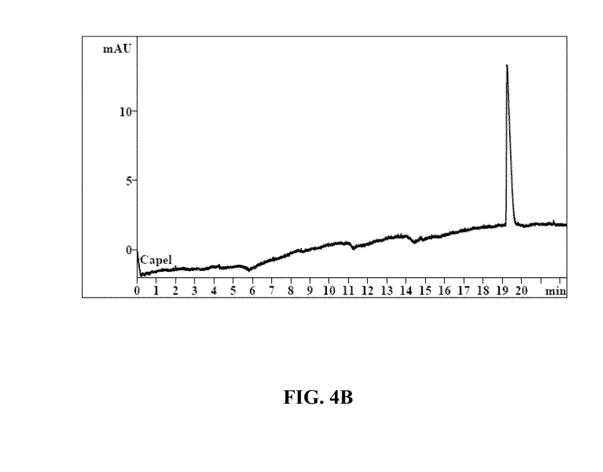

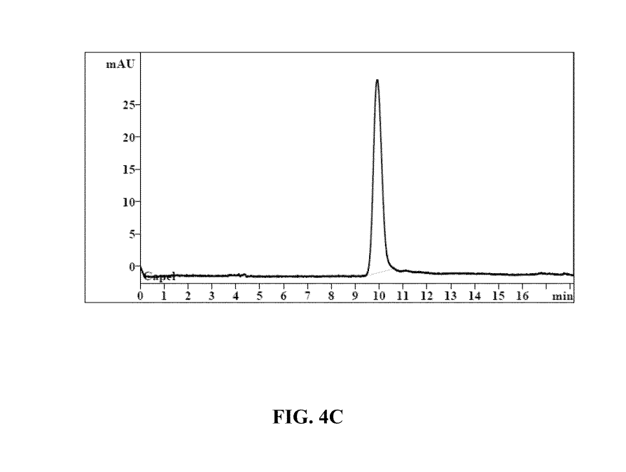

FIG. 4A shows an electropherogram of the Human Serum Albumin protein, according to an embodiment herein. With respect to FIG. 4A, it is clear that HSA has no detectable absorbance at this condition. The peak at 3 min is because of a little impurity in the column.

FIG. 4B shows an electropherogram of the Methotrexate drug, according to an embodiment herein. With respect to FIG. 4B, it is clear that the migration time of MTX drug is at time 20 min.

FIG. 4C shows an electropherogram of the Methotrexate-Human Serum Albumin (MTX-HSA) nanoparticles, according to an embodiment herein. With respect to FIG. 4C, the migration time of MTX-HSA nanoparticles is 10 min. Also this electropherogram proves immobilization of MTX onto the HSA as it has absorbance at 300 nm.

### Example 4

Investigation of MTX immobilization to the nanoparticles: A capillary zone electrophoresis system (CAPEL® 105) was applied to prove a covalent binding of the MTX to the NPs. Solutions of 3000 ppm of the HSA protein and MTX-HSA NPs and 400 ppm of the MTX in phosphate buffer (100 mM, pH 2) were prepared. The best wavelength for analysis of these solutions was 300 nm.

### Example 5

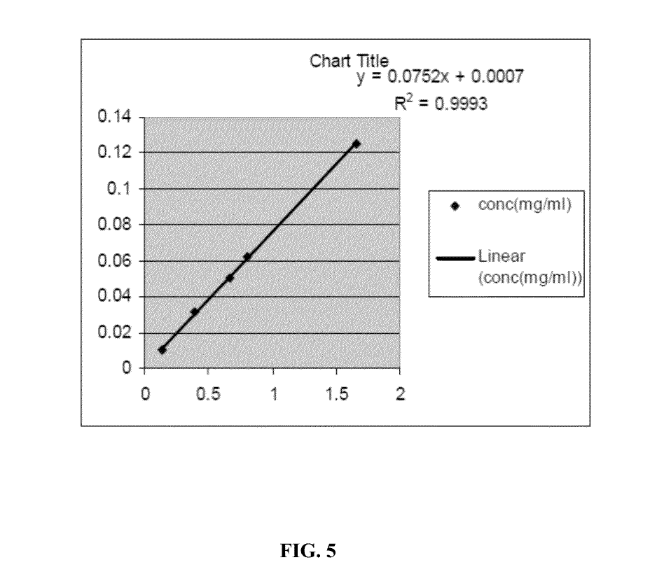

Determination of the drug loading efficiency: The calibration curve of the absorbance of the concentrations of MTX range from 0.01 to 0.125 mg/ml in 372 nm. FIG. 5 shows a calibration curve of absorbance of different concentrations of the MTX at 372 nm, according to the embodiments herein. The absorbance of a solution of 12.5 mg MTX-HSA NPs in 4 ml DMSO compared to the standard curve exhibited that drug containing of the 12.5 mg NPs is about 0.4596 μg and encapsulation efficiency is about 48%.

Encapsulation Efficiency %=Mass of encapsulated drugMass of initial drug×100

### Example 6

Conjugation of anti-MUC1 VHH to the MTX-HSA NPs: The Covalent attachment of VHH onto the NPs surface was achieved by using EDC and NHS reagents. A solution of 5 mg MTX-HSA NPs, 1 mg EDC and 2.5 mg NHS in 1 ml PBS (pH 7.4) was prepared. The reaction mixture was stirred at 200 rpm for 15 min at room temperature to activate carboxylate groups on the NP surface. To remove non-reacted EDC and NHS, the solution was ultrafiltered centrifuged by a 30 kDa ultrafilter device (Amicon® Ultra-30kDa) and centrifugation at 5000 g for 10 min. After the resuspension of the obtained nanoparticles in 1 ml of PBS (pH 7.4), 125 μg of anti-MUC1 VHH was introduced into the solution. The mixture was stirred at 200 rpm for 2 hrs at a room temperature. Finally, the solution was ultrafiltered centrifuged using a 30 kDa ultrafilter and centrifugation at 5000 g for 10 min. The filtered solution was stored for Bradford protein assay and the solution containing NPs was maintained for further analyses.

### Example 7

Particle size and zeta potential of the VHH-MTX-HSA NPs: 3 mg of the freeze-dried NPs were dispersed in PBS (pH 7.4). Then, hydrodynamic diameter and zeta potential of the particles were determined by a Zetasizer Nano ZS (Malvern Instruments, Worcestershire, UK).

### Example 8

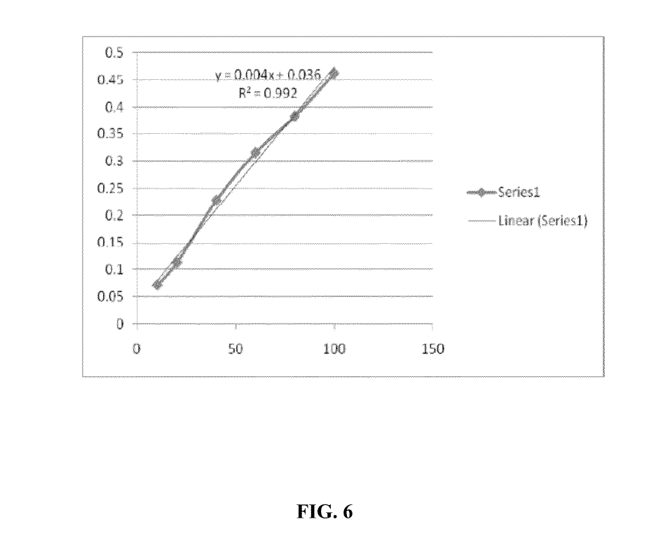

Investigation of the VHH conjugation efficiency to the MTX-HSA nanoparticles: For this purpose, a Bradford protein assay [20] was used. FIG. 6 shows a standard curve of the absorbance of the concentrations of the BSA ranging from 10 to 100 μg/ml at 595 nm, according to the embodiments herein. Then, the absorbance of 100 μg/ml of the filtered solution containing nanobody was investigated. Compared to the standard curve, an amount of the VHH in the solution was about 25 μg that means more than 80% of the initial nanobody is attached to the nanoparticles.

The Human serum albumin (HSA), as a natural polymer, is a very soluble versatile protein carrier for drug targeting and improving the pharmacokinetic profile of the drugs. The most important characteristic of the albumin is its ability to bind a great number of therapeutic drugs and molecules. Beside these properties, a specific accumulation in the tumor and inflamed tissues, a biodegradability and a lack of toxicity, make albumin an appropriate natural polymer for the drug delivery applications. The albumin accumulates in the malignant tissue due to the presence of leaky capillaries associated with an impaired lymphatic drainage system (through the EPR effect). All of the above-mentioned properties support the efficiency of this system as a drug carrier. In this respect, a drug delivery occurs by the passive targeting process. The system disclosed in the embodiment herein, has the capability of the active targeting. The monoclonal antibody that lack light chains was utilized and its single N-terminal domain (VHH or NANOBODY®) binds an antigen effectively. This kind of antibody has a lot of advantages over the conventional antibodies. It is very small (18 KD) in comparison with IgG (150 KD). Also the nanobodies are more soluble and stable against a heat-denaturation. In the embodiments herein, an anti-MUC1 nanobody as a targeting molecule is used against MUC1 glycoprotein that is over-expressed on the cancer cell surfaces in the breast adenocarcinoma and a number of epithelial tumors. Having these antibodies conjugated onto the nanoparticles, the anti-cancer drug is delivered specifically to the desired cell. The methotrexate molecules that are available on the nanoparticle surface and are antagonists of the folate have an extra role for an active internalization. The methotrexate molecules attach to the folate receptors on the cancer cell surfaces. Hydrodynamic size of the nanoparticles (100-200 nm), its negative zeta potential and an efficient drug loading capacity, are ideal for a targeted delivery system. The covalent attachment of the MTX to the nanoparticles ensures the site-specific release of the drug and therefore decreases its toxicity against the healthy cells. In summary, this system utilizes a novel cancer targeted drug carrier of which reaches and internalizes to the tumor cells through the passive and active strategies and leads to lower the cytotoxicity of the non-cancerous cells.

The foregoing description of the specific embodiments will so fully reveal the general nature of the embodiments herein that others can, by applying a current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A method of synthesizing a multi-mode cancer targeted nanoparticles comprising steps of:
preparing a plurality of nanoparticles, wherein the plurality of nanoparticles include a protein and a drug, wherein the protein and the drug are cross linked to each other, wherein the protein is Human Serum Albumin protein (HSA), and wherein the drug is selected from a group consisting of melphalan, doxorubicin, daunorubicin, gemcitabine and methotrexate, and wherein the drug is methotrexate (MTX), and wherein each of the plurality of nanoparticles including Human Serum Albumin protein and methotrexate is MTX-HSA; and
covalently conjugating monoclonal antibodies on a surface of the prepared plurality of nanoparticles, wherein the monoclonal antibody is a single N-terminal domain, and wherein the monoclonal antibody is anti MUC1 (mucin glycoprotein) nanobody (VHH), and wherein the multi-mode cancer targeted nanoparticles have an active and a passive tumor targeting capability, and wherein the multi-modal cancer targeted nanoparticles include the Human Serum Albumin protein, the methotrexate and the monoclonal antibody, and wherein the multi-modal cancer targeted nanoparticles comprising the Human Serum Albumin protein, the methotrexate and the monoclonal antibody is MTX-HSA-VHH.

2. The method according to claim 1, wherein the step of preparing the plurality of nanoparticles further comprises:
solubilizing the drug in a solvent with N-ethyl-N-(3-dimethylaminopropyl) carbodiimide HCl (EDC) and N-hydroxy succinimide (NHS) to form a first solution, wherein the solvent is Dimethyl sulfoxide (DMSO), wherein the Dimethyl sulfoxide (DMSO) acts as a denaturant;
incubating the first solution in a water bath at 60° C. for 10 min;
cooling the incubated solution to a room temperature;
adding the cooled incubated solution drop wise to a solution of protein while stirring at 400 rpm for 10 min to obtain a second solution, wherein the solution of protein is prepared by dissolving a Human Serum Albumin (HSA) in a phosphate buffer saline;
dialyzing the obtained second solution against a phosphate buffer saline (PBS) by using a dialysis tube for 24 h to obtain a dialyzed solution;
lyophilizing the dialyzed solution at −40° C. for 24 h to obtain a third solution containing the plurality of nanoparticles.

3. The method according to claim 1, wherein the step of covalently conjugating the monoclonal antibodies on a surface of the prepared plurality of nanoparticles further comprises:
solubilizing the obtained nanoparticles, N-ethyl-N-(3-dimethylaminopropyl) carbodiimide HCl (EDC) and N-hydroxy succinimide (NHS) in a phosphate buffer saline to form a reaction mixture;
stirring the reaction mixture at 200 rpm for 15 min at a room temperature;
performing an ultrafilter centrifugation of the stirred reaction mixture by a 30 kDa ultrafilter device;
performing a centrifugation of the stirred reaction mixture at 5000 g for 10 min to obtain the solubilized nanoparticles;
resuspending the obtained solubilized nanoparticles in 1 ml of a phosphate buffer saline (PBS) to obtain a solution, wherein the pH of the PBS is kept at 7.4;
introducing the monoclonal antibodies into the obtained solution to form a mixture, wherein the monoclonal antibodies are anti-MUC1 nanobodies, and wherein a concentration of the monoclonal antibodies is 125 μg;
stirring the formed mixture at 200 rpm for 2 hrs at room temperature;
performing an ultrafilter centrifugation of the stirred solution using a 30 kDa ultrafilter device;
performing a centrifugation of the stirred reaction mixture at 5000 g for 10 min to obtain the targeted protein based conjugated nanoparticles.

4. The method according to claim 1, wherein the protein is a denatured protein, and wherein the denatured protein is obtained using a denaturant, and wherein the denaturant is Dimethyl sulfoxide (DMSO).

5. The method according to claim 1, wherein the nanoparticles of MTX-HSA have a particle size of about 90-150 nm, and wherein the nanoparticles of VHH-MTX-HSA are spherical in shape with a hydrodynamic size in the range of 100-200 nm in diameter, and wherein the nanoparticles of VHH-MTX-HSA have a hydrodynamic size of 40 nm in diameter and wherein the nanoparticles of MTX-HSA have a zeta-potential of about −20 mv and wherein the nanoparticles of VHH-MTX-HSA have a zeta-potential in a range of −10 mv to −20 mv, and wherein the nanoparticles of VHH-MTX-HSA have a zeta-potential of −10 mv.

6. The method according to claim 1, wherein the drug has an efficiency of 48% and wherein the efficiency of monoclonal antibody conjugation is 80%, and wherein a drug loading efficiency of the nanoparticles is 48%, and wherein a monoclonal antibody conjugation efficiency is more than 80%.

7. A multi-mode cancer targeted nanoparticles comprising:
a plurality of cross linked nanoparticles of protein and drug molecules; and
covalently linked molecules of monoclonal antibodies, wherein the molecules of monoclonal antibodies are linked on a surface of the plurality of cross linked nanoparticles.

8. The nanoparticles according to claim 7, wherein the protein is Human Serum Albumin (HSA).

9. The nanoparticles according to claim 7, wherein the drug includes an anticancer drug containing carboxyl or primary amino functional groups, and wherein the drug is selected from a group consisting of melphalan, doxorubicin, daunorubicin, gemcitabine and methotrexate, and wherein the drug is methotrexate (MTX).

10. The nanoparticles according to claim 7, wherein the monoclonal antibody is a single N-terminal domain, and wherein the monoclonal antibody is anti MUC1 (mucin glycoprotein) (VHH).

11. The nanoparticles according to claim 7, wherein the protein is a denatured protein, wherein the denatured protein is obtained using a denaturant, and wherein the denaturant is Dimethyl sulfoxide (DMSO).

12. The nanoparticles according to claim 7, wherein the system has an active and passive tumour targeting capability.

13. The nanoparticles according to claim 7, wherein the cancer is a kind of cancer that over expresses mucin glycoproteins (MUC1) on cell surfaces, wherein the cancer is carcinoma.

14. The nanoparticles according to claim 7, wherein the nanoparticles of MTX-HSA have a particle size of about 90-150 nm, and wherein the nanoparticles of VHH-MTX-HSA are spherical in shape with a hydrodynamic size in the range of 100-200 nm in diameter, and wherein the nanoparticles of VHH-MTX-HSA have a hydrodynamic size of 40 nm in diameter

15. The nanoparticles according to claim 7, wherein the nanoparticles of MTX-HSA have a zeta-potential of about −20 mv and wherein the nanoparticles of VHH-MTX-HSA have a zeta-potential in a range of −10 mv to −20 mv, and wherein the nanoparticles of VHH-MTX-HSA have a zeta-potential of −10 mv.

16. The nanoparticles according to claim 7, wherein the drug has an efficiency of 48% and wherein the efficiency of monoclonal antibody conjugation is 80%.

17. The nanoparticles according to claim 7, wherein a drug loading efficiency of the nanoparticles is 48%.

18. The nanoparticles according to claim 7, wherein a monoclonal antibody conjugation efficiency is more than 80%.
